SEQUENCE LISTING (2 sequences)

SEQ ID NO: 1 - DNA, 12 bp, Artificial
  misc_feature: Synthetic Spiegelmer
ugagugacugac

SEQ ID NO: 2 - DNA, 47 bp, Artificial
  misc_feature: Synthetic Spiegelmer
cgugugaggcaauaaaacuuaaguccgaagguaaccaauccuacacg

(19) United States
(12) Patent Application Publication
(10) Pub. No.: US 2009/0281296 A1
(43) Pub. Date: Nov. 12, 2009

(54) PROCESS FOR THE PRODUCTION OF CONJUGATES FROM POLYSACCHARIDES AND POLYNUCELOTIDES

(75) Inventor: Klaus Sommemeyer, Rosbach (DE)

Correspondence Address: MDIP LLC, POST OFFICE BOX 2630, MONTGOMERY VILLAGE, MD, 20886-2630, US

(73) Assignees: Supramol Parenteral Colloid GMBH, Rosbach-Rodheim (DE); Noxxon Pharma AG, Berlin (DE)

(21) Appl. No.: 10/597,787

(22) PCT Filed: Feb. 8, 2005
(86) PCT No.: PCT/EP2005/001252

§ 371 Date: Sep. 5, 2007

(30) Foreign Application Priority Data
Feb. 9, 2004 (DE) 102004006249.8

(51) Int. Cl. C07H 21/00; C07H 5/06

(52) U.S. Cl. 536/22.1; 536/55.3

(57) ABSTRACT

The present invention relates to a process for the production of a conjugate from a polynucleotide and a polysaccharide comprising the steps: a) provision of an aldonic acid of the polysaccharide or of a derivative thereof; b) reaction of the aldonic acid with an alcohol derivative, preferably a carbonate derivative of an alcohol, to an aldonic acid ester, preferably to an activated aldonic acid ester; and c) reaction of the aldonic acid ester with the polynucleotide, wherein the polynucleotide exhibits a functional amino group, wherein the reaction of the aldonic acid with the alcohol derivative in step b) takes place in a dry aprotic polar solvent.

The present invention relates to a process for the production of a conjugate from a polynucleotide and a polysaccharide and to the conjugates obtainable according to a process of this type.

The conjugation of pharmaceutical active ingredients in particular of proteins with polyethyleneglycol derivatives (“PEGylation”) or polysaccharides such as dextrans or in particular hydroxyethyl starch (“HESylation”) has gained in importance in recent years with the increase in pharmaceutical proteins from biotechnological research.

The effects of a PEGylation or HESylation of pharmaceutically active compounds such as, for example, proteins consist, inter alia, in that by coupling the proteins to the above-mentioned polymers such as polyethyleneglycol (PEG) or hydroxyethyl starch (HES) their short biological half-life too low for the development of the full pharmaceutical potential can be specifically extended. The antigenic properties of proteins, however, can also be positively influenced by coupling. In the case of other pharmaceutical active ingredients, the water solubility can be considerably increased by coupling. Examples of the HESylation of pharmaceutical active ingredients are for example described in International Patent Application WO 02/080979 A2 or in International Patent Application WO 03/000738 A2.

More recent developments in the field of biological target molecules of high affinity binding oligonucleotides, such as for example the D-oligonucleotides referred to as aptamers or the L-oligonucleotides referred to as Spiegelmers, also use the possibilities of the conjugation to polymers such as polyethyleneglycol (B. Wlotzka et al. PNAS 13, vol. 99 (2002) pages 8898-8902) to change the pharmacokinetic profile and the bioavailability in an advantageous way.

HES is the hydroxyethylated derivative of the glucose polymer amylopectin present at over 95% in wax maize starch. Amylopectin consists of glucose units which are present in α-1,4-glycosidic bonds and exhibit α-1,6-glycosidic branchings. HES exhibits advantageous rheological properties and is currently used clinically as a volume replacer and for haemodilution therapy (Sommermeyer et al., Krankenhauspharmazie, vol. 8 (1987) pages 271-278 and Weidler et al., Arzneimittelforschung/Drug Res., 41 (1991) pages 494-498).

In DE 196 28 705 and DE 101 29 369 processes are described specifically for haemoglobin or amphotericin B, as to how coupling with hydroxyethyl starch in anhydrous dimethylsulphoxide (DMSO) can be carried out via the corresponding aldonic acid lactone of hydroxyethyl starch with free amino groups of haemoglobin or amphotericin B.

Since, particularly in the case of proteins, it is often not possible to work with anhydrous, aprotic solvents, either for reasons of solubility but also for reasons of denaturing the proteins, coupling processes with HES in a hydrous medium are also described in the literature. Thus for example International Patent Application PCT/EP 02/02928 discloses the coupling of hydroxyethyl starch selectively oxidising to aldonic acid at the reducing end of the chain by means of water-soluble carbodiimide EDC (1-ethyl-3-(3-dimethylaminopropyl)-carbodiimide). Very often, however, the use of carbodiimides is linked with disadvantages because carbodiimides very often cause inter- or intramolecular cross-linking reactions of the proteins as secondary reactions.

The present invention is based on the problem of providing a process for the production of a conjugate from a polynucleotide and a polysaccharide.

According to the invention, the problem is resolved in a first aspect by a process for the production of a conjugate from a polynucleotide and a polysaccharide comprising the steps of:
a) provision of an aldonic acid of the polysaccharide or of a derivative thereof;
b) reaction of the aldonic acid with an alcohol derivative, preferably a carbonate derivative of an alcohol, to an aldonic acid ester, preferably to an activated aldonic acid ester; and
c) reaction of the aldonic acid ester with the polynucleotide, wherein the polynucleotide exhibits a functional amino group,
characterised in that the reaction of the aldonic acid with the alcohol derivative in step b) takes place in a dry aprotic polar solvent.

In one embodiment, the solvent is selected from the group comprising dimethylsulphoxide, dimethylformamide and dimethylacetamide.

In one embodiment, the aldonic acid ester is purified and then is used in step c).

In an alternative embodiment, the reaction charge from step b) is used with the aldonic acid ester directly in step c).

In one embodiment, step c) is carried out at a pH range of 7 to 9, preferably 7.5 to 9 and more preferably 8.0 to 8.8.

In a preferred embodiment, step c) is carried out at a pH of approximately 8.4.

In one embodiment, the molar ratio of aldonic acid to alcohol derivative is approximately 0.9 to 1.1, preferably approximately 1.

In one embodiment, the alcohol is selected from the group comprising N-hydroxy-succinimide, sulphonated N-hydroxy-succinimide, phenol derivatives and N-hydroxy-benzotriazole.

In one embodiment, the polysaccharide is selected from the group comprising dextran, hydroxyethyl starch, hydroxypropyl starch and branched starch fractions.

In one embodiment, the polysaccharide is hydroxyethyl starch.

In a preferred embodiment, the hydroxyethyl starch exhibits a weight-averaged mean molecular weight of approximately 3,000 to 100,000 Dalton, preferably of approximately 5,000 to 60,000.

In a further preferred embodiment, the hydroxyethyl starch exhibits a number average of the mean molecular weight of approximately 2,000 to 50,000 Dalton.

In one embodiment, the hydroxyethyl starch exhibits a ratio of weight-averaged molecular weight to number average of the mean molecular weight of approximately 1.05 to 1.20.

In one embodiment, the hydroxyethyl starch exhibits a molar substitution of 0.1 to 0.8, preferably of 0.4 to 0.7.

In one embodiment, the hydroxyethyl starch exhibits a substitution sample expressed as the C2/C6 ratio of approximately 2 to 12, preferably of approximately 3 to 10.

In one embodiment, the polynucleotide is a functional nucleic acid.

In a preferred embodiment, it is provided that the functional nucleic acid is an aptamer or a Spiegelmer.

In one embodiment, it is provided that the polynucleotide exhibits a molecular weight of 300 to 50,000 Da, preferably 4,000 to 25,000 Da and more preferably 7,000 to 16,000 Da.

In one embodiment, it is provided that the functional amino group is a primary or secondary amino group, preferably a primary amino group.

In one embodiment, it is provided that the functional amino group is linked to a terminal phosphate of the polynucleotide.

In a preferred embodiment, it is provided that the functional amino group is linked to the phosphate group via a linker.

In one embodiment, it is provided that the functional amino group is a 5-aminohexyl group.

In a second aspect, the problem is resolved according to the invention by a conjugate of a polysaccharide and a polynucleotide, obtainable according to a process according to the first aspect of the present invention.

The present invention is based on the surprising knowledge that, from hydroxyethyl starch aldonic acids and aldonic acids of other polysaccharides, such as, for example, wax maize starch degradation fractions, in dry aprotic, polar solvents, such as, for example, dimethylacetamide (DMA), dimethylsulphoxide (DMSO) or dimethylformamide (DMF), with alcohols, in particular with the carbonates of alcohols, thus the diesters of carbon dioxide with alcohols, such as, for example, N-hydroxy-succinimides, the corresponding aldonic acid esters could be produced, which can be advantageously reacted in an aqueous medium with nucleophilic amino groups from polynucleotides to more stable amides. A saponification of the aldonic acid esters with water to the free aldonic acid and to the free alcohol occurs as secondary reaction.

Surprisingly, no activation of the hydroxyl groups of the anhydroglucose units thereby occurs, but instead specifically the activation of the carboxyl group of the aldonic acids, provided the molar ratios of the reactants is set in the region of 1:1.

The present invention in this respect turns away from the teaching described up until now in the prior art or is based on the knowledge that the different processes described in the prior art are not suitable for an efficient production of a conjugate of a polynucleotide and a polysaccharide.

Thus it was also surprisingly found by the present inventors that L-5′-amino-functionalised oligonucleotides cannot be reacted with HES aldonic acids via a carbodiimide (EDC)-mediated formation of an amide bond with the 5′ amino group, despite variations in the possible reaction parameters and reactant ratios.

It is in fact known that compounds containing phosphates and phosphate groups increase the loss of carbodiimides, often quite dramatically, but even large excesses of EDC did not, in the present case, lead to measurable reaction product (S. S. Wong, Chemistry of Protein Conjugation and Cross-Linking, CRC-Press, Boca Raton, London, New York, Washington D.C. (1993) page 199).

Furthermore, it is known that EDC can be used in aqueous medium to couple molecules containing amino functions to the terminal phosphate group of oligonucleotides forming a phosphoramidate bond. Under the reaction conditions thereby carried out, the internal phosphate groups do not react. In this way, 5′phosphate groups in particular can be specifically modified (Bioconjugate Techniques, Greg T. Hermanson, Academic Press, San Diego, New York, Boston, London, Sydney, Tokyo, Toronto (1996) page 52).

Furthermore, it was surprisingly found that also other established coupling methods which are described in the literature for hydroxyethyl starch derivatives, cannot be successfully used with 5-amino-functionalised L-oligonucleotides. Thus it is a common method to form the reactive acid imidazolide from the acid for the production of acid amides in the sense of a formation of an amide bond as intermediate stage and then, with this active acylation agent, carry out the reaction of the amine to the corresponding amide with the release of imidazole.

In the case of HES aldonic acid, production of the reactive HES imidazolide was successful. This however decomposed to imidazole and HES acid during the reaction in aqueous solution at all pH values and reactant ratios examined, without involving a coupling with the actually more nucleophilic 5-amino-functionalised polynucleotide.

As a further possibility, coupling was aimed for via an active hydroxysuccinimide ester of the HES acid, which was previously produced in anhydrous medium according to literature specifications either via EDC activation or by reaction of the HES lactone with hydroxysuccinimide in anhydrous medium. Neither of the two methods, however, was successful.

The reaction of HES via the only reducing end group with the amino-functionalised polynucleotide in the sense of a reductive amination was also unsuccessful, despite very long reaction times.

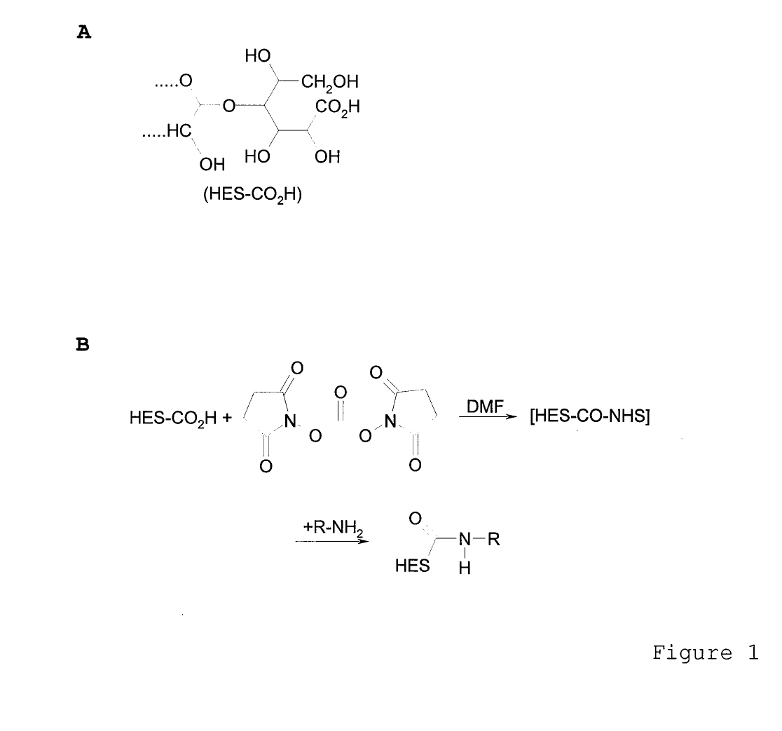

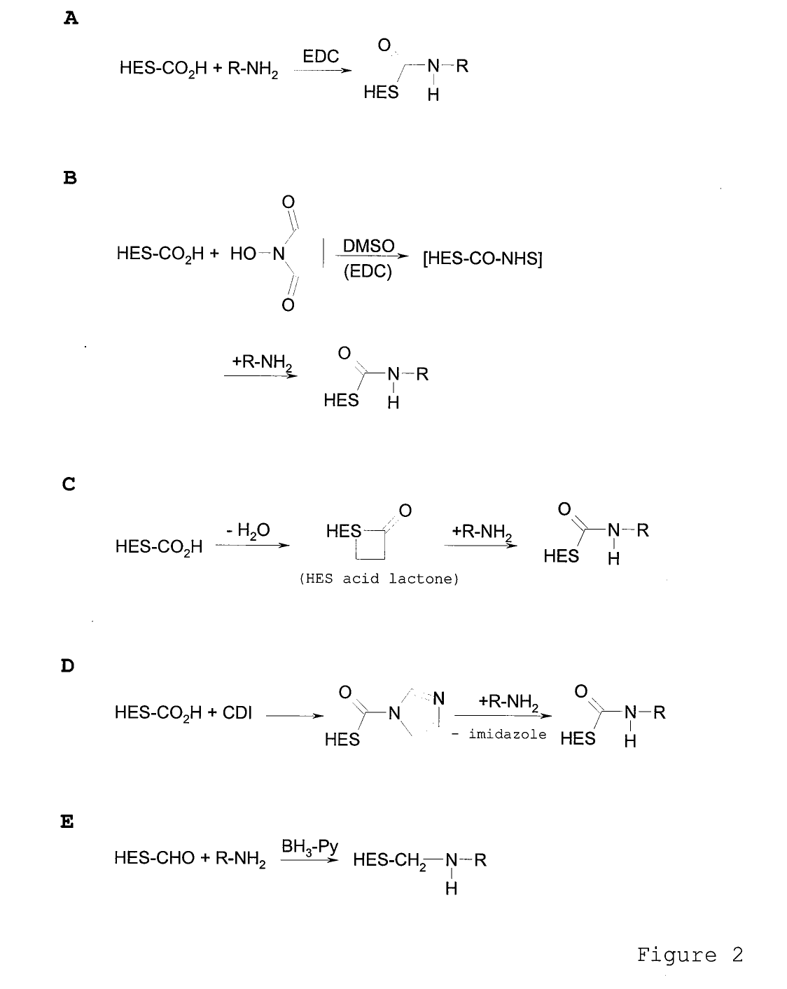

The reaction scheme for the production according to the invention of a conjugate from a polynucleotide and a polysaccharide is shown in FIG. 1, wherein FIG. 1A shows the structure of the aldonic acid group of the aldonic acid of the polysaccharide and FIG. 1B elucidates the course of the reaction. The reaction equations in FIG. 2, which are the subject matter of examples 4 to 14, summarise the unsuccessful attempts to produce a conjugate from a polynucleotide and a polysaccharide.

Although the process according to the invention is not, in principle, restricted to certain polysaccharides, hydroxyethyl starch is a particularly preferred polysaccharide. It is, however, also within the framework of the invention that other starch derivatives, such as e.g. hydroxyprolyl starch, are used. Likewise, within the framework of the present invention the hyperbranched starch fractions described in German Patent Application 102 17 994, in particular hyperbranched starch fractions with degrees of branching greater than 10 mol %, preferably greater than 10 mol % and smaller than 16 mol %, can be used.

HES is essentially characterised by the weight-averaged mean molecular weight Mw, the number average of the mean molecular weight Mn, the molecular weight distribution and the degree of substitution. The substitution with hydroxyethyl groups in ether bonds is thereby possible on carbon atoms 2, 3 and 6 of the anhydroglucose units. The substitution sample is thereby described as the ratio of the C2 to the C6 substitution (C2/C6 ratio). The degree of substitution can thereby be described as DS (English for “degree of substitution”) which refers to the content of the substituted glucose molecule of all glucose units or as MS (English for “molar substitution”) by which the average number of hydroxyethyl groups per glucose unit is denoted.

In scientific literature, as also herein, the molecular weight Mw in the unit kDalton is given as an abbreviation for hydroxyethyl starch together with the degree of substitution MS. Thus HES 10/0.4 denotes hydroxyethyl starch of the molecular weight Mw of 10,000 and of the degree of substitution MS of 0.4.

The production of the aldonic acid esters used according to the invention is performed by reacting the aldonic acid of the polysaccharide or its derivatives in dry, aprotic solvents such as, for example, dimethylformamide (DMF), dimethylsulphoxide (DMSO) or dimethylacetamide (DMA) and the carbonates of the alcohol component. The aldonic acids described herein are known from the prior art and can be produced for example in accordance with the disclosure of German Patent Application DE 196 28 705.

In the reaction of the aldonic acid with the alcohol derivative, in particular the carbonates of the respectively used alcohol, the molar ratio is approximately 0.9 to 1.1, preferably approximately 1.0, because with an excess of carbonate, as is provided by the alcohol derivative, OH groups of the polysaccharide are selectively activated and with less, excess acid functions are not reacted.

Particularly preferred alcohols within the framework of the present invention are N-hydroxy-succinimide, sulphonated N-hydroxy-succinimide, phenol derivatives and N-hydroxy-benzotriazole. Suitable phenol derivatives comprise, amongst others, chlorinated, fluorinated or nitrated compounds, wherein these can be activated once or several times, particularly by the afore-mentioned electrophilic groups. Correspondingly it is within the framework of the present invention to use mono- or polychlorinated phenols, mono- or polyfluorinated phenols or mono- or polynitrated phenols.

The aldonic acid esters used according to the invention can be precipitated from the solution in DMF by dry ethanol, isopropanol or acetone and purified or enriched by repeating the process several times. Such aldonic acid esters can then be used isolated in substance for coupling to polysaccharides. The solution of the reaction products in the inert apolar solvent can, however, also be reused directly, without isolation of the active aldonic acid ester for coupling to polysaccharides.

Within the framework of the present invention, in principle, that any type of polynucleotides is conjugated with a polysaccharide. The polynucleotide can thereby be produced from L-nucleosides or D-nucleosides or mixtures thereof, wherein these can individually or altogether exhibit other modifications, such as for example modifications to increase stability in biological systems. A modification of this type is for example the fluorination at position 2′ of the sugar constituent of the nucleotides or nucleosides. It is thereby also within the framework of the present invention that at least part of the sugar constituents of the nucleotides forming the polynucleotide can exhibit a sugar other than ribose or deoxyribose. Sugars of this type can, for example, be other pentoses, such as, for example, arabinose, but also hexoses or tetroses. Sugars of this type can also contain a nitrogen atom or sulphur atom, such as, for example in an aza- or a thiosugar, and/or the sugar content of the polynucleotide can be replaced at least partially by a morpholino ring. Furthermore, the polynucleotide can be developed at least partially as locked nucleic acids (LNA) or peptide nucleic acid (PNA). The OH groups of the molecule constituents forming the skeleton of the polynucleotide can be chemically modified by suitable NH₂, SH, aldehyde, carboxylic acid, phosphate, iodine, bromine or chlorine groups.

It is furthermore within the framework of the present invention that the polynucleotide is a ribonucleic acid or a deoxyribonucleic acid or combinations thereof, i.e. individuals or a group of nucleotides are present as RNA and the other nucleotides forming the nucleic acid are present as DNA and vice versa. The term L-nucleic acid is herein used synonymously with the term L-oligonucleotide or L-polynucleotide and refers, amongst others, to both L-deoxyribonucleic acid and also L-ribonucleic acid and combinations thereof, i.e. that individuals or a group of nucleotides are present as RNA and the other nucleotides forming the nucleic acid are present as DNA and vice versa. It is thereby also envisaged that in place of deoxyribose or ribose other sugars form the sugar component of the nucleotide. Furthermore is comprised the use of nucleotides with other modifications at position 2′ such as NH₂, OMe, OEt, Oalkyl, NHalkyl and the use of natural or unnatural nucleobases such as, for example, isocytidine and isoguanosine. It is thereby also within the framework of the present invention that the L-nucleic acid exhibits so-called abasic positions, i.e. nucleotides in which there is no nucleobase. Abasic positions of this type can be arranged both inside the nucleotide sequence of the L-nucleic acid and also at one or both ends, i.e. the 5′ and/or the 3′ end.

Furthermore, it is within the framework of the present invention that the polynucleotide is present as a single strand, wherein it is, however, also within the framework of the present invention that this is present as a double strand. Typically the polynucleotide used according to the invention is a single-strand L-nucleic acid which, however, as a result of its primary sequence can develop defined secondary structures and also tertiary structures. In the secondary structure, with a multiplicity of L-nucleic acids, double-strand sections are also present.

The conjugated nucleic acids described herein are preferably so-called Spiegelmers. As already mentioned at the beginning, Spiegelmers are functional L-nucleic acids or L-polynucleotides, i.e. such nucleic acids which bind to a target molecule or a part thereof, and are the result of contacting a nucleic acid library, in particular of a statistical nucleic acid library, with the target molecule.

Combinatorial DNA libraries are first of all produced for a selection process for the development of functional nucleic acids. As a rule, this is the synthesis of DNA oligonucleotides which centrally contain a range of 10-100 randomised nucleotides which are 5′ and 3′ terminal flanked by two primer bond regions. The production of combinatorial libraries of this type is, for example, described in Conrad, R. C., Giver, L., Tian, Y. and Ellington, A. D., 1996, Methods Enzymol., vol. 267, 336-367. Such a chemically synthesised single-strand DNA library can be converted via the polymerase chain reaction to a double-strand library which in itself can in fact be used for a selection. As a rule, however, a separation of the individual strands can be carried out with suitable methods so that an individual strand library which is used for the in vitro selection process if it is a DNA selection (Bock, L. C., Griffin, L. C., Latham, J. A., Vermaas, E. H. and Toole, J. J., 1992, Nature, vol. 355, 564-566) can again be achieved. It is, however, also possible to include the chemically synthesised DNA library directly in the in vitro selection. In addition, an RNA library can, in principle, be produced from double-strand DNA if a T7 promoter is introduced beforehand, thus via a suitable DNA-dependant polymerase, for example the T7 RNA polymerase. It is possible to produce libraries of 10¹⁵ and more DNA or RNA molecules using the process described. Each molecule from this library has a different sequence and consequently a different three-dimensional structure.

Via the in vitro selection process, it is then possible to isolate, from the library mentioned, through several cycles of selection and amplification and optionally mutation, one or several DNA molecules which exhibit a significant binding property against a given target. The targets can be, for example, viruses, proteins, peptides, nucleic acids, small molecules such as metabolism metabolites, pharmaceutical active ingredients or the metabolites thereof or other chemical, biochemical or biological components such as described for example in Gold, L., Polisky, B., Uhlenbeck, O. and Yarus, 1995, Annu. Rev. Biochem. vol. 64, 763-797 and Lorsch, J. R. and Szostak, J. W., 1996, Combinatorial Libraries, Synthesis, Screening and application potential, ed. Riccardo Cortese, Walter de Gruyter, Berlin. The process is carried out such that binding DNA or RNA molecules are isolated from the library originally used and are amplified after the selection step by means of polymerase chain reaction. In RNA selections, a reverse transcription should be pre-connected to the amplification step by polymerase chain reaction. A library enriched after a first selection round can then be used in a renewed selection round so that the molecules enriched in the first selection round have the chance to again succeed by selection and amplification and go with still more daughter molecules into a further selection round. At the same time, the polymerase chain reaction step opens up the possibility of introducing new mutations during amplification, e.g. by variation of the salt concentration. After a sufficient number of selection and amplification rounds, the binding molecules have succeeded. An enriched pool is thus produced, the representative of which can be isolated by cloning and then determined in its primary structure with the common methods of sequence determination of DNA. The sequences obtained are then checked for their binding properties with regard to the target. The process for the production of aptamers of this type is therefore referred to as the SELEX process and is described, for example, in EP 0 533 838, the disclosure of which is included herein by reference.

The best binding molecules can be shortened by shortening the primary sequences to their essential binding domain and represented by chemical or enzymatic synthesis.

A particular form of aptamers producible to such an extent are the so-called Spiegelmers which are substantially characterised in that they are constructed at least partially, preferably completely of the non-natural L-nucleotides. Processes for the production of Spiegelmers of this type are described in PCT/EP97/04726, the disclosure of which is included herewith by reference. The peculiarity of the process described therein lies in the production of enantiomer nucleic acid molecules, i.e. of L-nucleic acid molecules which bond to a native target, i.e. present in the natural form or configuration or a target structure of this type. The in vitro selection process described above is used to first of all select binding nucleic acids or sequences against the enantiomers, i.e. non-naturally occurring structure of a naturally occurring target, for example in the case where the target molecule is a protein, against a D protein. The binding molecules thus obtained (D-DNA, D-RNA or corresponding D-derivatives) are determined in their sequence and the identical sequence is then synthesised with mirror-image nucleotide building blocks (L-nucleotides or L-nucleotide derivatives). The mirror-image, enantiomer nucleic acids thus obtained (L-DNA, L-RNA or corresponding L-derivatives), so-called Spiegelmers, have, for reasons of symmetry, a mirror-image tertiary structure and consequently a binding property for the target present in the natural form or configuration.

The polynucleotides, in particular the functional nucleic acids such as aptamers or Spiegelmers, as obtained in the selection and shortening processes described herein, have a molecular weight of approximately 300 Da to 50,000 Da. Preferably these exhibit a molecular weight of 4,000 Da to 25,000 Da, more preferably 7,000 to 16,000 Da.

The target molecules described above, also referred to as target, can be molecules or structures, thus, for example, viruses, viroids, bacteria, cell surfaces, cell organelles, proteins, peptides, nucleic acids, small molecules such as metabolism metabolites, pharmaceutical active ingredients or the metabolites thereof or other chemical, biochemical or biological components.

According to the process according to the invention, the polynucleotide exhibits, preferably on a phosphate group of the polynucleotide, a nucleophilic group with which the aldonic acid ester reacts to form the conjugate. It is thereby particularly preferred within the framework of the present invention that this nucleophilic group is a functional amino group, preferably a primary amino group (NH₂ group). It is thus within the framework of the invention that the polynucleotide reacted with the aldonic acid ester contains a functional secondary amino group, an imino group.

It is however particularly preferred within the framework of the present invention that the nucleophilic group is a primary amino group which is preferably present bound on a phosphate group of the polynucleotide. The amino group is preferably present on the phosphate group at the 5′ or at the 3′ end, thus the terminal phosphate groups, of the polynucleotide. The amino group can thereby be bound in one embodiment either directly to the phosphate group or via a linker to the phosphate group. Linkers of this type are known from the prior art. Preferred linkers are alkyl radicals with a length of 1 to 8, preferably 2 to 6 C atoms. In particular, when using aldonic acid esters of N-hydroxy-succinimide, the nucleophilic groups furthermore present in the polynucleotide, such as the purine or pyrimidine base in the nucleic acids, are not reacted.

It is within the framework of the present invention that an oligonucleotide is used in place of a polynucleotide. In one embodiment, a polynucleotide, as used herein, is an oligonucleotide.

The present invention is illustrated by means of the following figures and examples from which other features, embodiments and advantages of the present invention are obtained. These show:

FIG. 1A the chemical structure of the aldonic acid group of the HES aldonic acid;

FIG. 1B a reaction scheme for the activation according to the invention of HES aldonic acid with a carbonate derivative of an alcohol to an aldonic acid ester and the reaction thereof with a polynucleotide bearing a functional amino group;

FIG. 2A the reaction scheme for the production of conjugates from a polynucleotide and HES aldonic acid in accordance with the prior art, in particular in accordance with examples 4-9;

FIG. 2B the reaction scheme for the production of conjugates from a polynucleotide and HES aldonic acid in accordance with the prior art, in particular in accordance with example 10;

FIG. 2C the reaction scheme for the production of conjugates from a polynucleotide and HES aldonic acid in accordance with the prior art, in particular in accordance with example 11;

FIG. 2D the reaction scheme for the production of conjugates from a polynucleotide and HES aldonic acid in accordance with the prior art, in particular in accordance with examples 12-13;

FIG. 2E the reaction scheme for the production of conjugates from a polynucleotide and HES in accordance with the prior art, in particular in accordance with example 14;

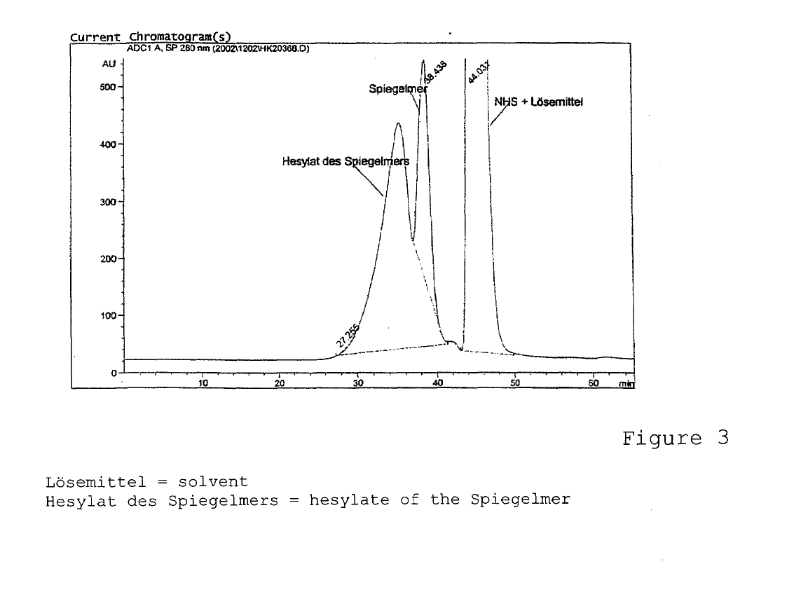

FIG. 3 a chromatogram of the results of a reaction charge for the HESylation of a Spiegelmer in accordance with the present invention, in particular in accordance with example 1;

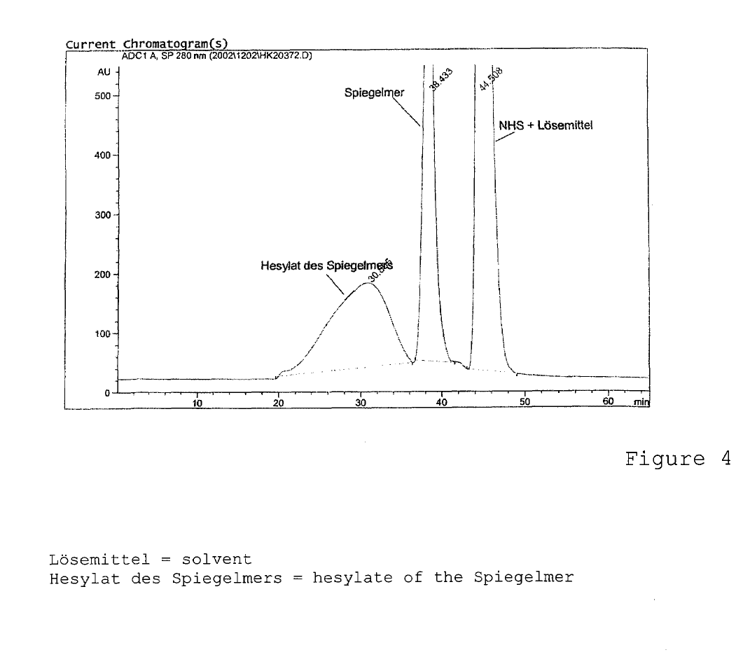

FIG. 4 a chromatogram of the result of a further reaction charge for the HESylation of a Spiegelmer in accordance with the present invention, in particular in accordance with example 1; and

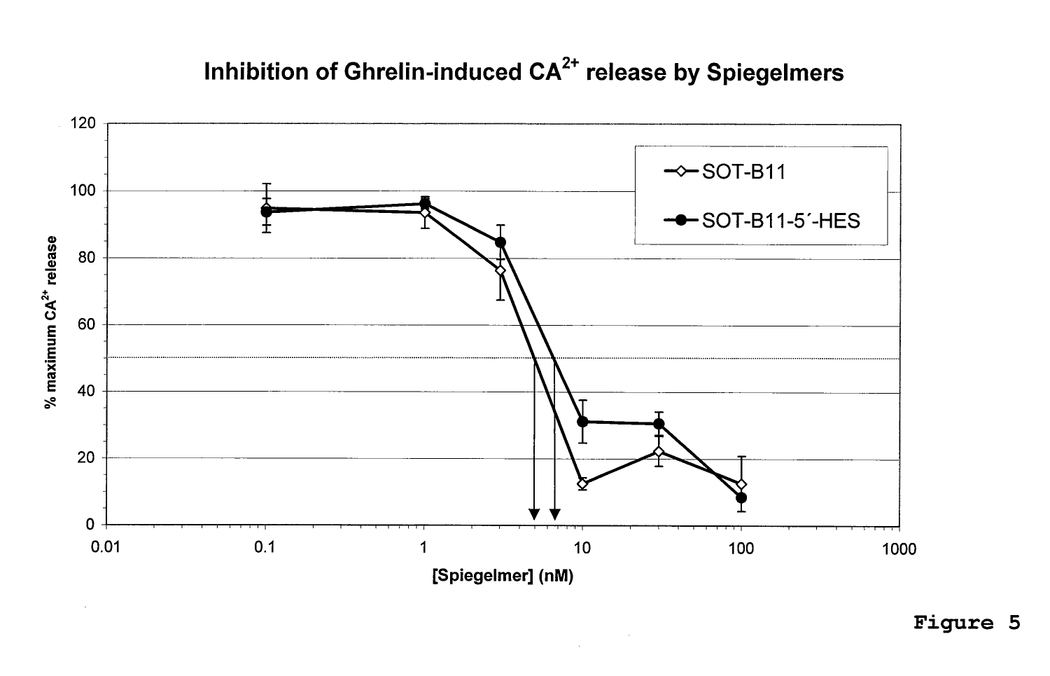

FIG. 5 a diagram of the inhibition caused by HESylated Spiegelmer or non-HESylated Spiegelmer of Ghrelin-induced calcium²⁺ release.

### EXAMPLE 1

### Production of a conjugate from a Spiegelmer and Hydroxyethyl Starch

### HESylate Used

HES 10/0.4 with the molecular parameters Mw 11092 D, MS 0.4 and C2/C6>8 which was oxidised to carboxylic acid at the reducing chain end, was used. A description of the production of the HES acid is disclosed for example in German Patent Application DE 196 28 705.

### Production of the NHS Ester

The N-hydroxy-succinimide ester of the HESylate was produced as follows:

0.2 g (0.05 mMol) anhydrous HES acid 10/0.4 is dissolved in 1 ml dry dimethylformamide and reacted with an equimolar quantity of N,N′-disuccinimidyl carbonate (12.8 mg) for 1.5 hours at room temperature.

### Production of the Spiegelmer HESylate

5 mg (corresponding to 1.3 μmol) 5′-aminohexyl functionalised RNA-Spiegelmer according to_Seq. ID. no. 1 are dissolved in 0.7 ml of a 0.3 molar dicarbonate solution with a pH of 8.4. The active ester, produced as described above, is added directly to this solution and reacted at room temperature for 2 hours.

The RNA-Spiegelmer exhibits the following sequence:

| | |
|---|---|
| 5′-aminohexyl-UGAGUGACUGAC-3′ | (SEQ. ID. NO. 1) |

Analysis of the Conjugate Produced in Such a Way

The conjugate is detected by low-pressure GPC. The analysis conditions thereby used were the following, the analysis result being shown in FIG. 3:
Column: Superose 12 HR 10/30, 300 mm×10 mm i.d. (Pharmacia, art. no. 17-0538-01)
Mobile solvent: Phosphate buffer pH 7.0 (27.38 mM Na₂HPO₄, 12.62 mM NaH₂PO₄, 0.2 M NaCl, 0.005% NaN₃ in Milli-Q water)
Flowrate: 0.4 ml/min
Detection: UV 280 nm
Running time: 70 min
Injection volume: 20 μl original charge

The reaction charge produced a yield of 62% (with perpendicular with reference to the chromatogram) or 77% with tailing peak evaluation.

The above reaction was carried out with another form of hydroxyethyl starch (50/0.7) which exhibited the following molecular parameters: Mw: 54110 D, MS: 0.7 and C2/C6: ˜5.

With an otherwise identical reaction procedure, a yield of 53% was obtained. The corresponding GPC chromatogram for the reaction product is shown in FIG. 4.

### EXAMPLE 2

### Increasing the Yield of the Conjugate

Based on the procedure described in example 1, the ratio of activated aldonic acid ester added in portions to test RNA-Spiegelmer was increased to 2:1 or 3:1. In the case of doubling of the ratio, a yield of more than 95% was obtained and with tripling of the excess of the activated aldonic acid ester, a virtually quantitative yield (>98 to 99%) was obtained.

### EXAMPLE 3

### Comparison of the Inhibition of the Ghrelin-Induced Calcium Release by Ghrelin-Binding HESylated and Non-HESylated Spiegelmers

Stably transfixed CHO cells which express the human receptor for Ghrelin (GHS—R1a) (obtained from Euroscreen, Gosselies, Belgium) are sown in a number of 5−7×10⁴ per well of a black 96-well microtitre plate with a clear base (Greiner) and cultivated overnight at 37° C. and 5% CO₂ in UltraCHO medium (Cambrex) which contains in addition 100 units/ml penicillin, 100 μg/ml streptomycin, 400 μg/ml Geneticin and 2.5 μg/ml Fungizone.

Non-HESylated and 5′-HESylated forms produced in accordance with example 1 of the Ghrelin-binding Spiegelmers with the internal reference SOT-B11 according to SEQ. ID no. 2 are incubated together with human or rat Ghreiin (Bachem) in UltraCHO Medium to which 5 mM probenecid and 20 mM HEPES have been added (CHO—U+), for 15-60 min at RT or 37° C. in a 0.2 ml “low profile 96-tube” plate. These stimulation solutions are prepared as ten times concentrated solutions in CHO—U+.

Sequence of SOT-B11:

(seq. ID. no. 2)

5′- CGU GUG AGG CAA UAA AAC UUA AGU CCG AAG
GUA ACC AAU CCU ACA CG -3′

Before charging with the calcium indicator dye Fluo-4, the cells are washed 1× with 200 μl respectively CHO—U+. 50 μl of the indicator dye solution (10 μM Fluo-4 (molecular probes), 0.08% Pluronic 127 (molecular probes) in CHO—U+ are then added and incubated for 60 min at 37° C. The cells are then washed 3× with 180 μl respectively CHO—U+. 90 μl CHO—U+ are then added per well.

Measurement of the fluorescence signals is carried out at an excitation wavelength of 485 nm and an emission wavelength of 520 nm in a Fluostar Optima multidetection plate reader (BMG).

The stimulation solutions are added to the cells for accurate analysis of the course of time for the changes in the calcium concentrations caused by Ghrelin. The wells of a vertical series of a 96 well plate are measured jointly for the parallel measurement of several samples. For this, three measured values are recorded first of all at an interval of 4 seconds to determine the baseline. Measurement is then discontinued, the plate removed from the reader and with a multi-channel pipette 10 μl of the stimulation solution from the “low profile 96-tube” plate in which the pre-incubation was carried out, added to the wells of the series to be measured. The plate is then again inserted in the machine and measurement continued (a total of 20 measurements 4 seconds apart).

From the measurement curves obtained, the difference between maximum fluorescence signal and fluorescence signal before stimulation is determined for each individual well and plotted against the concentration of Ghrelin or, in tests for the inhibition of calcium release with Spiegelmers, against the concentration of Spiegelmer.

To show the efficiency of the HESylated Spiegelmers, Ghrelin receptor-expressing cells were stimulated with 5 nM Ghrelin or Ghrelin which has been pre-incubated together with different quantities of HESylated or non-HESylated Spiegelmer. The fluorescence signals measured were standardised to the signals which were obtained without Spiegelmers. The HESylated Spiegelmer inhibits the Ghrelin-induced Ca⁺⁺ release with an IC₅₀ of approx. 6.5 nM whereas the non-HESylated Spiegelmer inhibits with an IC₅₀ of approx. 5 nM. The result is shown in FIG. 5.

### EXAMPLE 4

### Production of Conjugates from a Polynucleotide and HES Aldonic Acid Using Processes According to the Prior Art

0.25 g HES 10/0.4 aldonic acid (62.5 μmol) is dissolved with stirring in 10 mL water. 9.95 mg (2.5 μmol) RNA-Spiegelmer according to SEQ. ID. no. 1 are added to the solution at room temperature. 50 mg N-ethyl-N′-(3-dimethylaminopropyl)-carbodiimide hydrochloride (261 μmol) dissolved in 1 mL water are then added with stirring in portions over 2 hours at room temperature. A pH of 5 is kept constant by addition of hydrochloric acid or sodium hydroxide solution. When the reaction is complete, the charge is further stirred for another 2 hours at room temperature. Checking of the reaction charge via low-pressure GPC gave a reaction conversion of the Spiegelmer used of less than 1%.

### EXAMPLE 5

### Production of Conjugates from a Polynucleotide and HES Aldonic Acid Using Processes According to the Prior Art

150 mg EDC are added to a mixture of HES 10/0.4 aldonic acid and RNA-Spiegelmer according to seq. ID. no. 1 as in example 4 over 3 hours at room temperature and with stirring. No reaction conversion could be determined by analytical low-pressure GPC.

### EXAMPLE 6

### Production of Conjugates from a Polynucleotide and HES Aldonic Acid Using Processes According to the Prior Art

1.0 g HES 10/0.4 aldonic acid (240 μmol) are dissolved with stirring in 10 mL water with heat. After cooling to room temperature, 10 mg of the RNA-Spiegelmer are added to the charge according to seq. ID. no. 1. 50 mg EDC (260 μmol) dissolved in 1 mL water are then added in portions with stirring over 2 hours at room temperature, the pH being kept constant at 5 with hydrochloric acid or sodium hydroxide solution.

After a further 2 hours reaction time, the charge is analysed by means of low-pressure GPC. No reaction product could be detected.

### EXAMPLE 7

### Production of Conjugates from a Polynucleotide and HES Aldonic Acid Using Processes According to the Prior Art

The charge according to example 6 was repeated, wherein in this case 100 mg EDC were added over 3 hours.

When the reaction was complete, no reaction product could be detected.

### EXAMPLE 8

### Production of Conjugates from a Polynucleotide and HES Aldonic Acid Using Processes According to the Prior Art

Examples 6 and 7 were repeated at pH values of 4.0 and 6.0.

No reaction product could be detected in the two reaction charges by means of low-pressure GPC.

### EXAMPLE 9

### Production of Conjugates from a Polynucleotide and HES Aldonic Acid Using Processes According to the Prior Art

Example 4 was repeated at reaction temperatures of 4° C. and 37° C.

In both cases, no reaction product was detected.

### EXAMPLE 10

### Production of Conjugates from a Polynucleotide and HES Aldonic Acid Using Processes According to the Prior Art

303.7 mg (2.6 mmol) succinimide and 0.502 g HES 10/0.4 aldonic acid (0.125 mmol) are dissolved in 10 mL dry dimethylsulphoxide (DMSO) at room temperature.

50 mg EDC (0.25 mmol) are then added and the charge stirred overnight.

5 mg (corresponding to 1.3 μmol) RNA-Spiegelmer according to seq. ID. no. 1 are dissolved in 10 mL water and the pH set at 8.5 with sodium hydroxide solution or dissolved in 10 mL 0.3 molar bicarbonate buffer of pH 8.4.

5 mL respectively of the above-named dimethylsulphoxide solution are added to the two partial charges, the pH of the aqueous solution of the first partial charge being kept constant at pH 8.5 by the addition of sodium hydroxide solution.

The charges were stirred overnight at room temperature. The analytical low-pressure GPC did not produce any reaction product in the two partial charges.

### EXAMPLE 11

### Production of Conjugates from a Polynucleotide and HES Aldonic Acid Using Processes According to the Prior Art

300 mg (2.6 mmol) succinimide are dissolved in 10 mL dry dimethylsulphoxide (DMSO) and 0.5 g (0.125 mmol) dried HES 10/0.4 aldonic acid added at 80° C. overnight to form the corresponding lactone. The charge reacts overnight at 70° C.

The solution is then added at room temperature to 5 mL of a solution of 5 mg RNA-Spiegelmer according to seq. ID. no. 1 in 10 mL 0.3 molar bicarbonate buffer of pH 8.4 and stirred for 4 hours at room temperature. No reaction product was found by means of analytical low-pressure GPC.

### EXAMPLE 12

### Production of Conjugates from a Polynucleotide and HES Aldonic Acid Using Processes According to the Prior Art

5.0 g HES 10/0.4 aldonic acid (1.2 mmol) are dissolved in 30 mL dry dimethylformamide (DMF). 195 mg (1.2 mmol) carbonyl diimidazole (CDI) are added to the solution and stirred for 2 hours at room temperature.

5 mg RNA-Spiegelmer according to seq. ID. no. 1 are dissolved in 5 mL water. 10 mL of the above-named solution of imidazolyl-HES aldonic acid 10/0.4 are added to this solution and the pH set at 7.5 with sodium hydroxide solution. After stirring at room temperature overnight, the charge was examined for reaction product by means of low-pressure GPC. Only traces of reaction product were determined.

### EXAMPLE 13

### Production of Conjugates from a Polynucleotide and HES Aldonic Acid Using Processes According to the Prior Art

5 mg RNA-Spiegelmer according to seq. ID. no. 1 are dissolved in 12.5 mL 0.3 M bicarbonate buffer of pH 8.4. The charge was cooled with ice water to 0° C. and mixed with 8.5 mL of the solution of HES 10/0.4 aldonic acid-imidazolyl in DMF mentioned in Example 12. After 2 hours at 0° C. and a further 2 hours at room temperature, the charge was examined for reaction product. No product could be detected.

### EXAMPLE 14

### Production of Conjugates from a Polynucleotide and HES Using Processes According to the Prior Art

1 g HES 10/0.4 (0.25 mmol) are dissolved in 5 mL H₂O with heat. 10 mg (corresponding to 2.5 μmol) RNA-Spiegelmer according to seq. ID. no. 1 are added to the solution after cooling and the pH set at 7.5 with sodium hydroxide solution. 200 μl borane-pyridine complex (Sigma-Aldrich) are then added and the charge stirred at room temperature in the dark for 10 days. The charge is then examined for any reaction products by low-pressure GPC. Only a conversion of <3% based on the Spiegelmer used could be detected.

The features of the invention disclosed in the preceding description, the claims and the drawings can be essentially both individual and also in any combination to perform the invention in its different embodiments.

1. Process for the production of a conjugate from a polynucleotide and a polysaccharide comprising the steps:
a) provision of an aldonic acid of the polysaccharide or of a derivative thereof;
b) reaction of the aldonic acid with an alcohol derivative, preferably a carbonate derivative of an alcohol, to an aldonic acid ester, preferably to an activated aldonic acid ester; and
c) reaction of the aldonic acid ester with the polynucleotide, wherein the polynucleotide exhibits a functional amino group,
characterised in that the reaction of the aldonic acid with the alcohol derivative in step b) takes place in a dry aprotic polar solvent.

2. Process according to claim 1, characterised in that the solvent is selected from the group comprising dimethylsulphoxide, dimethylformamide and dimethylacetamide.

3. Process according to claim 1 or 2, characterised in that the aldonic acid ester is purified and is then used in step c).

4. Process according to claim 1 or 2, characterised in that the reaction charge from step b) is used with the aldonic acid ester directly in step c).

5. Process according to one of claims 1 to 4, characterised in that step c) is carried out at a pH range of 7 to 9, preferably 7.5 to 9 and more preferably 8.0 to 8.8.

6. Process according to claim 5, characterised in that step c) is carried out at a pH of approximately 8.4.

7. Process according to one of claims 1 to 6, characterised in that the molar ratio of aldonic acid to alcohol derivative is approximately 0.9 to 1.1, preferably approximately 1.

8. Process according to one of claims 1 to 7, characterised in that the alcohol is selected from the group comprising N-hydroxy-succinimide, sulphonated N-hydroxy-succinimide, phenol derivatives and N-hydroxy-benzotriazole.

9. Process according to one of claims 1 to 8, characterised in that the polysaccharide is selected from the group comprising dextran, hydroxyethyl starch, hydroxypropyl starch and branched starch fractions.

10. Process according to claim 9, characterised in that the polysaccharide is hydroxyethyl starch.

11. Process according to claim 10, characterised in that the hydroxyethyl starch exhibits a weight-averaged mean molecular weight of approximately 3,000 to 100,000 Dalton, preferably of approximately 5,000 to 60,000.

12. Process according to one of claims 10 or 11, characterised in that the hydroxyethyl starch exhibits a number average of the mean molecular weight of approximately 2,000 to 50,000 Dalton.

13. Process according to one of claims 10 to 12, characterised in that the hydroxyethyl starch exhibits a ratio of weight-averaged molecular weight to number average of the mean molecular weight of approximately 1.05 to 1.20.

14. Process according to one of claims 10 to 13, characterised in that the hydroxyethyl starch exhibits a molar substitution of 0.1 to 0.8, preferably of 0.4 to 0.7.

15. Process according to one of claims 10 to 14, characterised in that the hydroxyethyl starch exhibits a substitution sample expressed as the C2/C6 ratio of approximately 2 to 12, preferably of approximately 3 to 10.

16. Process according to one of claims 1 to 15, characterised in that the polynucleotide is a functional nucleic acid.

17. Process according to claim 16, characterised in that the functional nucleic acid is an aptamer or a Spiegelmer.

18. Process according to one of claims 1 to 17, characterised in that the polynucleotide exhibits a molecular weight of 300 to 50,000 Da, preferably 4,000 to 25,000 Da and more preferably 7,000 to 16,000 Da.

19. Process according to one of claims 1 to 16, characterised in that the functional amino group is a primary or secondary amino group, preferably a primary amino group.

20. Process according to one of claims 1 to 19, characterised in that the functional amino group is bound to a terminal phosphate of the polynucleotide.

21. Process according to claim 20, characterised in that the functional amino group is bound to the phosphate group via a linker.

22. Process according to one of claims 1 to 21, characterised in that the functional amino group is a 5-aminohexyl group.

23. Conjugate of a polysaccharide and a polynucleotide, obtainable according to a process according to one of claims 1 to 22.
